(19) United States
(12) Patent Application Publication
(10) Pub. No.: US 2010/0119709 A1
(43) Pub. Date: May 13, 2010

(54) Organic colorants and compositions containing same

(76) Inventor: Sean Michael Ragan, Austin, TX (US)

Correspondence Address: Sean Michael Ragan, 13004 S. Madrone Trail, Austin, TX, 78737, US

(21) Appl. No.: 12/291,321

(22) Filed: Nov. 8, 2008

(51) Int. Cl. C07D 265/34; C07C 245/08; C09D 7/12; C08K 5/3437; C08K 5/23; B05D 3/00; B05D 5/00; C09B 67/36; B65D 83/14

(52) U.S. Cl. 427/256; 544/99; 564/271; 106/287.21; 106/18.32; 524/89; 106/287.3; 524/190; 427/384; 427/395; 427/394; 427/397; 8/605; 8/565; 222/394

(57) ABSTRACT

Compositions and methods useful for imparting coloration to various substrates including plastics, paper, wood, fabrics, fibers and textiles. Materials comprising water-soluble colorants including an amidine group and a chromophore moiety are contacted with a substrate, and the substrate permitted to dry to provide substantially water-proof coloration on the substrate. In one embodiment an ink, coating, or dye is an aqueous composition comprising one or more amidines as provided herein, which is contained within a vessel that includes a gas at an effective level of pressure to enable the gas to react with an amidine as herein provided, to provide and maintain the amidine substantially in its protonated form prior to application of the aqueous composition to a desired substrate.

### TECHNICAL FIELD

The present disclosure relates generally to colorants useful in a wide variety of end-use applications. More particularly, it relates to organic molecules having controllably-variable water solubility, and inks, dyes, and coatings compositions comprising the colorants provided herein.

### BACKGROUND

The statements in this section merely provide background information related to the present disclosure and may not constitute prior art.

The use of dyes for imparting coloration to man-made and naturally-occurring articles dates back thousands of years, and includes the use of plant-derived pigments, dyes, and the like either alone or in admixture with other substances for providing both decorative and functional coloration to items which include without limitation pottery, jewelry, clothing, home furnishings, body paints, and architectural constructs.

Attendant to advances in the organic chemistry arts in the 19^{th} and 20^{th} centuries, workers have been able to synthesize, isolate, and bulk-manufacture scores of novel organic-molecule based dyestuffs and compositions of matter containing such dyestuffs, including printing inks and other coatings compositions. Such compositions often include solvents, either aqueous or non-aqueous which contain one or more dyestuffs in admixture with other components. In addition to solutions comprising colorant-imparting compositions, emulsions and suspensions including either or both inorganic and organic materials have been employed as well.

### SUMMARY

Compositions useful for imparting coloration to inks, dyes, coatings, and substrates, comprising at least one material described by the formulae selected from the group consisting of:

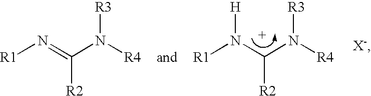

wherein at least one of R1, R2, R3, and R4 is a chromophore moiety, and the group(s) R1, R2, R3, and R4 which are not a chromophore moiety are each independently selected from the group consisting of: hydrogen and any C1-C24 hydrocarbyl group; and X⁻ is an anion present, inter alia, for charge balance. In a preferred embodiment, X⁻ is bicarbonate ion, HCO₃⁻. In another embodiment, X⁻ is an alkyl carbonate ion corresponding to the structure ROCO₂⁻ in which R is any C1 to C12 alkyl group. Such materials may be provided by combining an alkylcarbonic acid with the base form of an chromophoric material as provided herein having an amidine in its molecular structure, which may include bubbling carbon dioxide through an alcoholic solution of the amidine. However X⁻ may be any monovalent anion, including: chloride, bromide, iodide, nitrate, sulfate, hydrogen sulfate, bicarbonate, alkyl carbonates (ROCO₂⁻ in which R is any C1 to C12 alkyl group), phosphate, mono- and poly-hydrogen phosphates, borate, silicate, and any anion which provides charge balance for the protonated form of the amidine(s) provided herein, or from which carbon dioxide may be liberated.

Also provided are methods for imparting coloration to a substrate. A method according to one embodiment comprises providing an aqueous solution comprising at least one material having the structure:

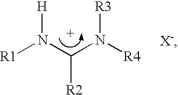

wherein at least one of R1 R2, R3, and R4 is a chromophore moiety, and non-chromophore moiety(ies) R1, R2, R3, and R4, when present, are each independently selected from the group consisting of: hydrogen and any C1-C24 hydrocarbyl group; and X⁻ is selected from the group consisting of: bicarbonate ion and any C1-C12 alkyl carbonate ion. The aqueous solution is applied to a substrate and dried. The drying may be accomplished by applying a current of ambient or heated air to the substrate, or by permitting the substrate to cure under ambient conditions until the solution is dried.

### BRIEF DESCRIPTION OF THE DRAWINGS

In the annexed drawing, provided for illustrative purposes only and not to be construed as delimitive of the present invention:

FIG. 1 shows an article of manufacture representative of a system comprising a material provided in accordance with the present disclosure.

### DETAILED DESCRIPTION

The present disclosure provides materials that are useful as color-imparting components in a wide variety of end-use compositions, which include without limitation: dyes, inks, and coating products such as paints and varnishes. In one embodiment, a material provided herein can be thought of in general as being an amidine, having the general structure:

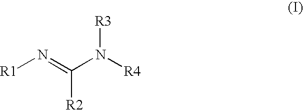

wherein at least one of R1, R2, R3, and R4 is a chromophore moiety, and the non-chromophore moiety(ies) R1, R2, R3, and R4, when present, are each independently selected from the group consisting of: hydrogen and any C1-C24 hydrocarbyl group. In one embodiment, R1 is a chromophore moiety and R2, R3, R4 are independently selected from the group consisting of: hydrogen and any C1-C24 hydrocarbyl group. In another embodiment, R1 and R2 are chromophore moieties and R3, R4 are independently selected from the group consisting of: hydrogen and any C1-C24 hydrocarbyl group. In another embodiment, R1, R2, and R3 are chromophore moieties and R4 is independently selected from the group consisting of: hydrogen and any C1-C24 hydrocarbyl group. In another embodiment, R1, R2, R3, and R4 are all chromophore moieties. Any one or more, or all, of the groups R1, R2, R3, R4 in the above structural formula or its protonated form may be chromophore moieties, including all possible combinations and permutations in which one, two or three or all of R1, R2, R3, R4 are chromophores which impart observable color to the molecule.

The term “hydrocarbyl”, when referring to a substituent or group in the present disclosure and the claims appended hereto is used in its ordinary sense, which is well-known to those skilled in the art. Specifically, it means a group having a carbon atom directly attached to the remainder of the molecule and having predominantly hydrocarbon character. Examples of hydrocarbyl substituents or groups include: (1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical); (2) substituted hydrocarbon substituents, that is, substituents containing non-hydrocarbon groups which, in the context of this disclosure, do not alter the predominantly-hydrocarbon substituent (e.g., halo (especially chloro and fluoro), hydroxy, alkoxy, mercapto, alkylmercapto, nitro, nitroso, and sulfoxy); (3) hetero substituents, that is, substituents which, while having a predominantly hydrocarbon character, in the context of this disclosure, contain other than carbon in a ring or chain otherwise composed of carbon atoms. Heteroatoms include sulfur, oxygen, nitrogen, and encompass substituents such as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group. All hydrocarbyl groups, whether straight-chain, branched, aliphatic, aromatic or groups containing any combination of the foregoing are useful within the meaning of R1, R2, R3, and R4 in the above, generally hydrophobic, structural formula (I) above for the amidines of this disclosure, subject to the proviso that no portion of a hydrocarbyl radical or group present is detrimentally-reactive under the conditions present with any labile bonds also present in the amidine.

A chromophore moiety suitable as acting as one or more of R1, R2, R3, and R4 in reference to structural formula (I) above may comprise or be derived from any known chromophoric materials which are colored, impart coloration, display any color, or appear colored by visual observation. Chromophoric materials suitable for providing a chromophore moiety include without limitation materials falling within the general classes of materials anthraquinones, acridines, azo dyes, bis-azo dyes, thiazine dyes, oxazine dyes, cresyl derivatives, and aminofluoresceins. Some examples include, without limitation the following, with CAS numbers being provided in parentheticals for some of these materials: Fast Garnet GBC, Nile Red, Acridine Yellow G (base) (135-49-9), 3,6-diaminoacridine, 9-aminoacridine, Basic Blue 47 (12217-43-5), Pararosaniline base, Rosaniline base, Bismarck Brown R base (Solvent Brown 12), Bismarck Brown Y base (Solvent Brown 41), 6-butoxy-2,6-diamino-3,3′-azodipyridine (617-19-6), Solvent Orange 3, Disperse Blue 1 (2475-45-8), Disperse Orange 3 (730-40-5), Disperse Yellow 9 (6373-73-5), Fast Blue BB Base (120-00-3), Fast Blue RR (6268-05-9), Fast Garnet GBC Base (97-56-3), Fast Red ITR Base (97-35-8), Fast Red Violet LB Base (121-22-2), Fast Violet B (99-21-8), Fat Brown RR (6416-57-5), Mordant Brown 4 (6247-27-4), Mordant Brown 48 (6232-53-7), alpha-Naphthyl Red (131-22-6), New Fuchsin base, Fuchsin base, 4-Phenylazoaniline (60-09-3), and Thionin base.

In preferred embodiments, the chromophoric material that comprises at least one of R1, R2, R3, R4 or from which such R groups is/are derived comprises an organic material having a primary amino group. In one embodiment, the chromophoric organic material has a single primary amino group. In another embodiment, the chromophoric organic material has two primary amino groups present in its molecular structure. In another embodiment, the chromophoric organic material has three primary amino groups present in its molecular structure, such as the case when pararosaniline is used as a reactant starting material in a reaction as herein described to provide a material having three amidine groups attached to it. In other embodiments, the chromophoric organic material has more than three primary amino groups present in its molecular structure.

In one embodiment, a material as described by formula (I) above is prepared by reaction of an amine substituent that is present on a chromophoric material, such as according to one non-limiting example, Nile Red, with the dimethyl acetal of N,N-dimethylacetamide (“DMADA”):

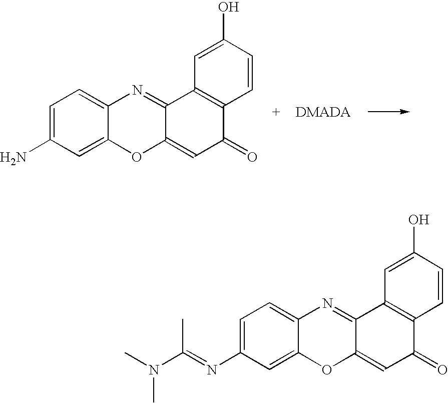

which may be effected by combining the reactants and heating to about 60° C. in the absence of a solvent. In analogous fashion, chromophore moieties derived from other known colored materials or coloring agents having a primary amino group as part of their molecular structure can be provided as a substituent for R1 in the structural formula (I) above. For cases where any one or more of R2, R3, R4 are desired to comprise a chromophore moiety in the structural formula (I) above, this may be accomplished by use of an acetal having a selected or desired chromophore in the desired position(s) on the acetal as a raw material. Accordingly, a process useful for providing a material according to one embodiment of this disclosure may be generalized as:

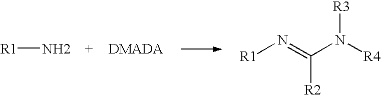

wherein R2, R3, R4 are each a methyl group. However, hydrogen and different hydrocarbyl substituents for R2, R3, and R4 in structure (I) above may be readily and selectively provided, by way of example, by employing different acetals as reactants. For example, use of the di-ethyl acetal of N,N-dimethyl propionamide as a reactant provides a product according to (I) wherein R2 is ethyl, and R3, R4 are both methyl. Use of the di-ethyl acetal of N,N-di-n-butyl butyramide provides a product according to (I) wherein R2 is n-propyl, and R3, R4 are both n-butyl. By employment of any desired or selected acetal of N,N-di-substituted amides, the identities of the substituents R2, R3, and R4, which are chosen to not be chromophore moieties, are readily controlled.

In another non-limiting example, Fast Garnet GBC is combined with dimethyl acetal of dimethylacetamide:

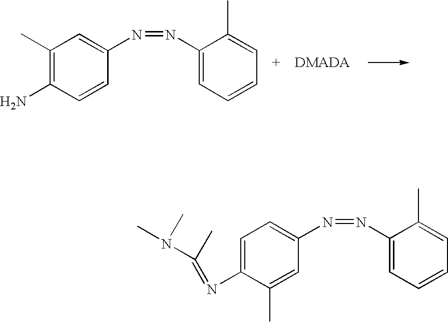

to provide a material in accordance with this disclosure, in which the R1 group is derived from the organic colorant Fast Garnet GBC. To effect the reaction, equimolar amounts of Fast Garnet GBC and DMADA were combined in a round-bottomed flask and set in an oil bath maintained at 60° centigrade, with stirring. The methanol formed was distilled off, the residue taken up in tetrahydrofuran and sparged with argon for ½ hour. The THF was removed and the material dried overnight under high vacuum at room temperature. Thin-layer chromatography of the material using 2:1 hexanes:EtOAc yielded two mobile phases, one of which co-eluted with the starting material, and a baseline spot that showed a strong blue color upon reaction with ninhydrin. One gram of the residue was taken up in methanol, adsorbed onto dry silica, and dried overnight under high vacuum. The adsorbed mixture was loaded onto a CombiFlash Companion® automated flash chromatograph and separated into three distinct fractions per the unit's built-in UV detector, and the longest-eluting fraction was identified by mass spectroscopy and proton NMR as being essentially pure amidine of Fast Garnet GBC, the product in the above reaction, at an 87.9% overall yield. The structure of the material was confirmed by X-ray diffraction of its hydrochloride salt.

In some embodiments, chromophoric materials having more than one amino group in their molecular structure are used as reactants analogously to the monoamines shown in the reactions above. In such embodiments an appropriate stoichiometric amount of acetal reactant is provided for the chosen reactant and the reaction is carried out in a conventional fashion.

Purification of an amidine function containing composition according to this disclosure may be carried out using means known in the art, including solvent extraction techniques, chromatography, reduced-pressure distillation, and molecular distillation.

One feature of materials represented by and described in reference to structure (I) above is that the materials are capable of existence in a protonated form. One of the protonated forms, which includes a counterion, may be provided by admixture of a material according to (I) with carbonic acid, represented as CO₂ and H₂O, according to the equilibrium:

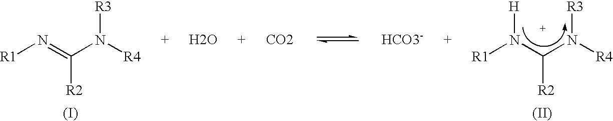

in which R1, R2, R3, and R4 are as previously defined. The material having generally-hydrophilic structure (II), being ionic and bearing a single positive charge, has increased solubility as compared to the generally-hydrophobic starting material (I) in aqueous solutions. Although the reaction equilibrium immediately above might be construed to imply that the change from structure (I) to structure (II) is the result of acid-base proton transfer, the mechanisms underlying reactions described in the present disclosure shall not necessarily be construed as being bound to this or any other single theory.

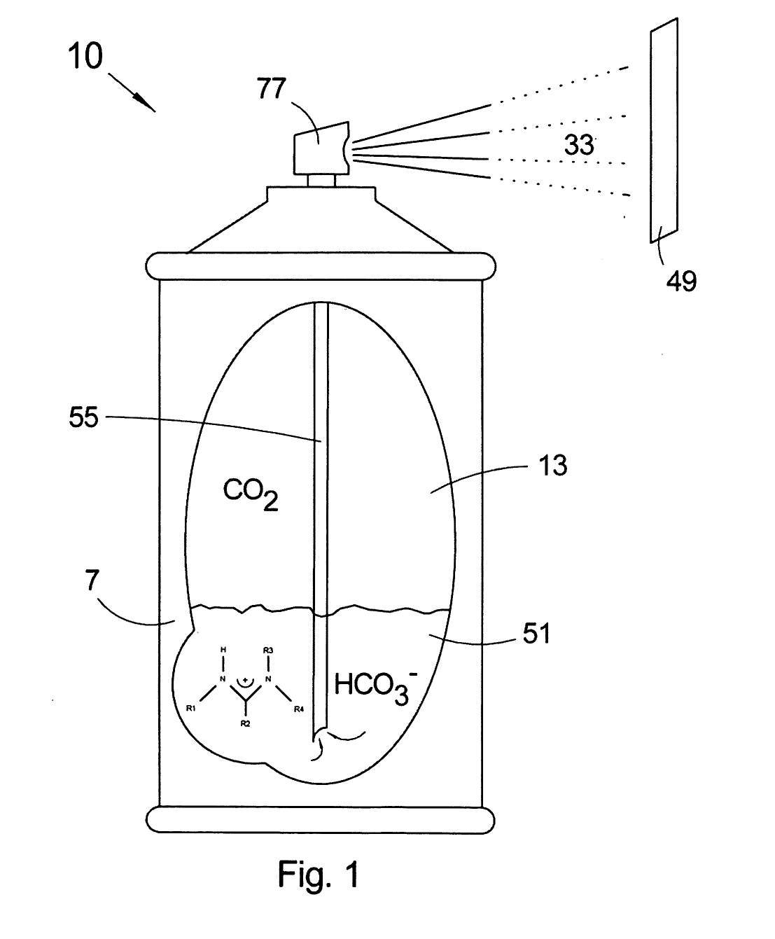

In some embodiments, advantage is taken of the difference in aqueous solubility of the materials defined by the structural forms (I) and (II). For example, FIG. 1 shows an article of manufacture, which is an aerosol container 10, in which is contained a liquid phase 51 and a gaseous phase 13. In some embodiments, the liquid phase 51 comprises an aqueous solution that includes a material according to structure (II) above, the protonated form of (I), and the gaseous phase 13 comprises carbon dioxide at a pressure above ambient pressure. In one embodiment, the carbon dioxide gas is substantially-pure, having purity of at least about 90% and the pressure of said substantially-pure carbon dioxide gas is any pressure in the range of between about 100 kiloPascals and about 42,000 kiloPascals. In another embodiment, the gaseous phase is a gas mixture comprising carbon dioxide and at least one other material which is a gas at standard temperature and pressure. For embodiments employing a gas mixture, the partial pressure of carbon dioxide gas in the gas mixture is preferably any partial pressure in the range of between about 10 kiloPascals and about 40,000 kiloPascals. In some embodiments, the gas mixture comprises nitrogen, at any partial pressure in the range of between about 10 kiloPascals and 40,000 kiloPascals.

As one non-limiting example, the liquid phase 51 may be an aqueous solution comprising a material according to structure (II) in which the R1 group is derived from the organic colorant Fast Garnet GBC, and the pressure of carbon dioxide in the gaseous phase is three atmospheres of pressure. Depressing the spray nozzle 77 in conventional fashion enables the carbon dioxide present to expand and force some of the liquid phase into the conveyance tube 55 from which the liquid phase is conveyed to a nozzle 77, which functions as an outlet conduit, to enable the liquid phase to be discharged from the container 10 in the form of a stream, mist, or aerosol 33 as desired, onto the surface of a substrate 49. In some embodiments the outlet conduit may be a piezo-electrically actuated ink jet. In other embodiments the outlet conduit comprises a hose or other tubing. In further embodiments, the outlet conduit comprises a conventional spraygun; however, any known device having an orifice through which an ink, dye, colorant, or coating composition may be applied to a substrate is suitable for use in applying a composition or material provided herein to a substrate. Once the material from the liquid phase 51 has contacted the substrate 49, by LeChatlier's principle the material described by (II) in the equilibrium above changes forms, owing to loss of CO₂, and the colorant disposed on the substrate 49 which previously existed in the form of structure (II) when it resided within the container 10 is accordingly caused to exist in the form of structure (I). Since structure (I) is not generally soluble in water, the present disclosure thus provides a method for dispensing a composition of matter comprising a colorant from an aqueous solution, which colorant becomes insoluble in water following its being permitted to “cure” by the loss of CO₂ and water when bicarbonate is selected as the counterion present with a material having structure (II).

While the foregoing example described a situation in which the material according to structure (II) in which the R1 group is derived from the organic colorant Fast Garnet GBC, the liquid phase 51 in such an arrangement may comprise any one or more colorants provided by structure (II) wherein R1, R2, R3, and R4 may each independently be any group, moiety, or substituent as previously set forth. Such materials may be present in the liquid phase 51 at any concentration in the range of between about one milligram (0.001 g) per liter, up to the solubility limit of the particular material chosen, including all concentrations and ranges of concentrations therebetween. Typically the total amount of pigments and colorants in an ink, dye or coating composition provided according to this disclosure is in the range of between about 1% and about 30% by weight based on the total weight of the composition. In some embodiments, the total amount of pigments and colorants in an ink, dye or coating composition provided according to this disclosure is in the range of between about 2% and about 10% by weight based on the total weight of the composition.

The liquid or solvent present as or in the liquid phase 51 of a coating, dye, or ink composition according to the present disclosure is sometimes referred to in the art as being the carrier medium. A carrier medium present in a composition that includes a material as provided herein can be either aqueous, or non-aqueous. When aqueous, the carrier medium is water or comprises a mixture of water and at least one organic solvent which is soluble in water to an appreciable extent. One preferred water-soluble organic solvent comprises one or more polyhydric alcohols. Polyhydric alcohols include ethylene glycol, propylene glycol; diols such as butanediol, pentanediol. Glycols and glycol esters are also useful, and include those such as glycerol, propylene glycol laurate; polyalkyl glycols such as polyethylene glycol; and lower alkyl ethers of polyhydric alcohols, such as ethylene glycol monomethyl ether, ethylene glycol mono-ethyl ether and ethylene glycol mono-butyl ether.

Other suitable water-soluble organic solvents include lower alcohols and all their isomers having fewer than about 8 carbon atoms per molecule such as methanol, ethanol, propanol, iso-propanol; ketones such as for example acetone; ethers such as for example, dioxane; esters such as ethyl acetate, propyl acetate, and lactams such as 2-pyrrolidone.

However, in a preferred embodiment, the solvent or carrier medium comprises water only, which is beneficial in providing environmentally-friendly coatings products having essentially a zero volatile organic carbon content, which reduces stress on both personnel using or handling the compositions as provided herein, and other life forms in the environment.

The amount of solvent present in a coating, dye or ink composition according to this disclosure is any amount in the range of between about 50% to about 99.8%, preferably about 70% to about 99.8% based on total weight of the composition. Selection of a particular composition as being suitable for a given final-use formulation depends on requirements of the specific application, such as desired surface tension and viscosity, the selected pigment(s), drying time of the composition, and type of substrate onto which the composition is intended to be disposed, as is generally recognized or appreciated by those skilled in this art.

Other colorant material(s) may be present in a liquid phase 51 composition as provided herein, including both organic and inorganic, which other colorant material(s) do not cause an adverse change in solubility of the materials having structure (II) present in the composition. Candidate colorant materials include without limitation: carbon blacks, titanium oxides, iron oxides, azo pigments (such as azo lakes, insoluble azo pigments, condensed azo pigments, and chelate azo pigments), polycyclic pigments (such as phthalocyanine pigments, perylenes and perylene pigments, anthraquinone pigments, quinacridone pigments, dioxazine pigments, thioindigo pigments, isoindolinone pigments, and quinophthalone pigments), lake pigments (such as base dye lakes, and acid dye lakes), nitro pigments, nitroso pigments, and aniline black daylight fluorescent pigments. Other pigments may also be used such as those that are dispersed in a water phase or those whose surfaces have been treated with a surfactant or a polymeric dispersing agent (such as graphite).

For cases where a colorant other than one described by structure (II) is chosen to be present in the liquid phase 51, known dispersants may be advantageously employed to be present in the liquid phase, in any effective-dispersing amount.

A surfactant may be optionally present in a composition as provided herein to modify the surface tension of the composition to control penetration of the composition into paper or other substrates. Examples of surfactants include nonionic, amphoteric, anionic, zwitterionic, and cationic surfactants, and those of ordinary skill in this art are aware of the surfactants employed in this field. Other additives such as binders (resins), biocides, humectants, mordants, chelating agents, viscosity modifiers, fillers, and de-foamers may also be present in a composition according to the disclosure. Optionally, acrylic and non-acrylic polymers may be added to improve properties such as water fastness and smear resistance. These may be solvent based, emulsions, or water soluble polymers. Materials within the foregoing classes that are known in the art are suitable as ingredients in compositions according to this disclosure.

A composition according to the present disclosure may be suitably prepared by combining the various components and mixing them in a suitable mixing device, which can include an ordinary kitchen blender, or other conventional mixing equipment.

In one embodiment, a material conforming to structure (I) as shown and described herein is mixed with water in a suitable containment vessel with gentle agitation, and gaseous carbon dioxide is sparged into the mixture, causing the material conforming to structure (I) to take on its cationic form of structure (II), bicarbonate ion generated in such a process serving as a counter-ion for charge balance and its ability to lose CO₂ after such material is subsequently applied to a substrate. However, acids other than carbonic acid are also useful for providing cationic forms of the chromophoric amidines of structure (II), such as when purifying a material as provided herein according to structure (I), the hydrochloride salt, as one non-limiting example, may be produced by combination of a material conforming to structure (I) as shown and described herein with aqueous hydrochloric acid to aid separation when it is desired to employ solvent extraction techniques in a purification scheme employing an aqueous phase and an organic phase. Such purification that uses solvent extraction may also include any other selected acidic material which enables partition.

Although one apparatus for dispensing a composition as provided herein was shown and described in reference to FIG. 1, a composition as provided herein may be applied using other conventional spraying or dispersing equipment, including hand-held spray guns employed in applying paints to articles such as automobiles which are typically fed using compressed air. Proceeding according to one embodiment of this disclosure, the air is substituted by a gaseous mixture comprising carbon dioxide present at at least 10% on a molar basis based on the total gas composition. However, a gaseous composition comprising any amount of carbon dioxide between about 2% and about 100% on a molar basis based on the total composition of the gaseous mixture may be employed, with an amount of carbon dioxide between about 50% and about 100% on a molar basis based on the total composition of the gaseous mixture being preferred. Such gaseous mixtures may comprise air or other gases which are inert towards and do not react with a composition according to the disclosure, including without limitation nitrogen and the noble gases.

In other embodiments, a composition as provided herein may be contained within and dispensed onto a substrate by means of what is commonly known as an “inkjet cartridge”, including without limitation those exemplified by U.S. Pat. Nos. 7,325,915; 7,258,431; 7,029,108; 7,011,397; 7,008,038; 6,742,879; 6,328,423; 5,631,683; 5,574,490; and 5,434,603, and other known inkjet cartridges, including piezo-electrically actuated ink jets having no cartridge portion. In one embodiment, an ink composition comprising a material conforming to structure (II) above is caused to be present within an inkjet cartridge itself, which contains an atmosphere comprising carbon dioxide at ambient or super-atmospheric pressure in the headspace above the ink or within the ink compartment(s) for maintenance of the colorant in the form represented by structure (II).

One advantage of compositions as provided herein over those of the prior art, is that prior art inks employed in ink-jets and ink-jet cartridges remain soluble even after they are disposed on a substrate, which means that if the substrate encounters moisture, the ink is susceptible to smearing. Compositions according to the present disclosure, on the other hand, undergo a change in solubility following their being disposed on a substrate, which change makes them essentially resistant to moisture-induced smearing or bleeding. Hence, compositions as provided herein may be generally regarded as being water-proof or substantially water-proof under normal conditions, this disclosure generally providing water-proof coatings, inks, dyes, etc. from a water-based solution.

Suitable substrates onto which a composition as provided herein may be applied include without limitation: plastics, paper, wood, fabrics, fibers and textiles.

For applications in which a composition as provided herein is to be employed as a dye for textiles, including various natural and synthetic fabrics and fibers, one embodiment involves immersing raw fabric or fibers into an aqueous solution comprising a material conforming to structure (II) in a suitable dyeing vessel in the presence of an effective amount of carbon dioxide to maintain the colorant in the form of structure (II) substantially throughout the process. Following a desired exposure time in the solution, the fabric or fibers are removed from the dyeing vessel and the fabric or fibers are rinsed if desired, dried, optionally with the aid of heat, to enable carbon dioxide to evolve and convert the colorant having structure (II) to the form of structure (I), which is generally insoluble in water, thus providing colorfast fabric or fibers.

Examples provided below are illustrative of materials and methods provided according to some embodiments of the present disclosure, and shall not be construed as being delimitive hereof in any way.

### EXAMPLE I

### Fast Garnet GBC Amidine

Equimolar amounts of Fast Garnet GBC and N,N-dimethylacetamide dimethylacetal (9.284 and 5.488 g, respectively) were combined in a round-bottom flask in an oil bath preheated to 60° C., and held for 20 min. Methanol side-product was removed on Rotavap and remaining brown/crimson oil taken up in THF and bubbled through with Ar for 30min. THF was then removed on Rotavap, followed by overnight drying under hard vacuum to remove solvent traces. Yielded 10.672 g (87.9% theoretical). Upon addition of the product to a biphasic system of mineral oil and water, color partitions selectively into oil layer until CO₂ is bubbled through, at which point the aqueous layer begins to turn orange. Bubbling Ar through a control flask did not cause this effect.

### EXAMPLE II

### GBC Amidine

87.04 mg Fast Garnet GBC were weighed into a small glass bottle and heated in an oil bath to 60° C. 0.13 mL dimethylacetamide dimethylacetal were added via syringe. After 2 h, heat was removed and the bath allowed to cool to room temperature. The next day, residue was taken up in 5 mL 1-octanol, shaken and left over the weekend, at which time solution was poured into 50 mL de-ionized water in a small separatory funnel and bubbled vigorously with CO₂ for 45 min. The resulting emulsion was allowed to stand, separating overnight, at which point the aqueous fraction was removed and sampled for mass spectrometry. The oil layer was washed four times with 50 mL de-ionized water until the washings came off mostly clear. Another 50 mL diH₂O was added and the biphasic system bubbled through with CO₂, again, for 45 min. Product was recovered from the pooled aqueous layers.

### EXAMPLE III

### Disperse Blue 1 Amidine

100.56 mg Disperse Blue 1 were weighed into a small, glass bottle and heated to 60° C. on an oil bath. 0.44 mL dimethylacetamide dimethylacetal were added via a syringe. After 2 h, heat was removed and the bath allowed to cool to RT. The next day, residue was dried overnight on the hi-vac and, the following morning, taken up in 5 mL 1-octanol, shaken, and left overnight to dissolve. About a week later, the octanol solution was washed with 6×50 mL de-ionized H₂O until washings were clear. First and last washings sampled for mass spectrometry. Octanol solution was layered above 50 mL clean de-ionized H₂O and bubbled with CO₂ for 5 min, at which point a marked color change between oil and water layers was observed and recorded photographically and by sampling each layer.

### EXAMPLE IV

### Pararosaniline Amidine

99.90 mg freebase pararosaniline were weighed into a small glass bottle and heated to 60° C. in an oil bath. 0.29 mL dimethylacetamide dimethylacetal were added via syringe. After 2 h, the heat was removed and the bath allowed to cool to RT. Residue was dried overnight on high vacuum and then taken up in 5 mL 1-octanol, shaken, and left overnight to dissolve.

### EXAMPLE V

### Chrysodine G Amidine

102.01 mg Chrysodine G were weighed into a small glass bottle and heated on an oil bath to 60° C. 0.23 mL dimethylacetamide dimethylacetal were added via syringe. 2 h later, heat was removed and bath allowed to cool to room temperature. Residue was dried overnight on the high vacuum and taken up in 5 mL 1-octanol, shaken, and left overnight to dissolve.

Provided below are examples of ink compositions according to various embodiments of the disclosure, in which the amounts of the components present in each example are expressed in parts by weight. The amidine components are present as the protonated form shown in structure (II) above, in these examples in their CO₂ form; however it is to be appreciated that anions other than bicarbonate may be present, such as when alkyl carbonates are selected to be present for charge balance or desirable reaction kinetics, per the foregoing.

Ink Composition #1

| | |
|---|---|
| Amidine of Fast Garnet GBC (protonated form) | 1% |
| Cc2ccccc2/N═N/c1ccc(/N═C(\C)N(C)C)c(C)c1 | |
| Water | 99% |

Ink Composition #2

| | |
|---|---|
| Amidine of Fast Garnet GBC (protonated form) | 15% |
| Water | 85% |

Ink Composition #3

| | |
|---|---|
| Amidine of Fast Garnet GBC (protonated form) | 1% |
| TRITON X-100 ® surfactant | 1% |
| Water | 98% |

Ink Composition #4

| | |
|---|---|
| Amidine of Fast Garnet GBC (protonated form) | 15% |
| TRITON X-100 ® surfactant | 6% |
| Water | 79% |

Ink Composition #5

| | |
|---|---|
| Amidine of Mauveine A (protonated form) | 1% |
| Cc1ccc(cc1)Nc2ccc3nc5cc(C)c(N)cc5[n+](c3c2)c4ccccc4 | |
| TRITON X-100 ® surfactant | 1% |
| Ethylene Glycol | 10% |
| Water | 88% |

Ink Composition #6

| | |
|---|---|
| Amidine of Mauveine A (protonated form) | 15% |
| TRITON X-100 ® surfactant | 6% |
| Ethylene Glycol | 10% |
| Water | 69% |

Ink Composition #7

| | |
|---|---|
| Mono-amidine of Blue 36 (protonated form) | 1% |
| CC(C)Nc3ccc(\N═C(/C)N(C)C)c2c3C(═O)c1ccccc1C2═O | |
| TRITON X-100 ® surfactant | 1% |
| PROXEL GXL ® biocide | 0.40% |
| Ethylene Glycol | 10% |
| Water | 87.6% |

Ink Composition #8

| | |
|---|---|
| Mono-amidine of Solvent Blue 36 (protonated form) | 15% |
| TRITON X-100 ® surfactant | 1% |
| PROXEL GXL ® biocide | 0.40% |
| Ethylene Glycol | 10% |
| Water | 73.6% |

Ink Composition #9

| | |
|---|---|
| Amidine of Nile Red A (protonated form) | 1% |
| CN(C)C(\C)═N\c3ccc4\N═C1C(═C/C(═O)c2ccccc12)\Oc4c3 | |
| TRITON X-100 ® surfactant | 1% |
| PROXEL GXL ® biocide | 0.40% |
| GlASCOL F110 ® fixative | 2% |
| Ethylene Glycol | 10% |
| Water | 85.6% |

Ink Composition #10

| | |
|---|---|
| Amidine of Nile Red A(protonated form) | 15% |
| TRITON X-100 ® surfactant | 1% |
| PROXEL GXL ® biocide | 0.40% |
| GlASCOL F110 ® fixative | 2% |
| Ethylene Glycol | 10% |
| Water | 71.6% |

Ink Composition #11

| | |
|---|---|
| Amidine of Nile Red A (protonated form) | 1% |
| Sodium dodecyl sulfate | 1% |
| PROXEL GXL ® biocide | 0.40% |
| GlASCOL F110 ® fixative | 2% |
| Ethylene Glycol | 10% |
| Water | 85.6% |

Ink Composition #12

| | |
|---|---|
| Nile Red A Amidine (protonated form) | 15% |
| Sodium dodecyl sulfate | 1% |
| PROXEL GXL ® biocide | 0.40% |
| GlASCOL F110 ® fixative | 2% |
| Ethylene Glycol | 10% |
| Water | 71.6% |

Ink Composition #13

| | |
|---|---|
| Mono-amidine of Solvent Blue 36 (protonated form) | 1% |
| Sodium dodecyl sulfate | 1% |
| PROXEL GXL ® biocide | 0.40% |
| Ethylene Glycol | 10% |
| Water | 87.6% |

Ink Composition #14

| | |
|---|---|
| Mono-amidine of Solvent Blue 36 (protonated form) | 15% |
| Sodium dodecyl sulfate | 1% |
| PROXEL GXL ® biocide | 0.40% |
| Ethylene Glycol | 10% |
| Water | 73.6% |

Consideration must be given to the fact that although the subject matter present in this disclosure has been provided concerning at least one preferred embodiment, there exists a likelihood that modifications and alterations which are equivalent or substantially-equivalent under this disclosure may become apparent to others upon considering, understanding, and appreciating the foregoing specification and the claims appended hereto. Such modifications and alterations are within this disclosure, which shall only be limited by the broadest interpretation of the scope of the claims which follow.

I claim:

1. A composition useful for imparting coloration to inks, dyes, coatings, and substrates, comprising at least one material described by the formulae selected from the group consisting of: 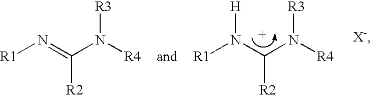
wherein at least one of R1, R2, R3, and R4 is a chromophore moiety, and non-chromophore moiety(ies) R1, R2, R3, and R4, when present, are each independently selected from the group consisting of: hydrogen and any C1-C24 hydrocarbyl group; and wherein the anion X⁻, when present, is any counter ion suitable for contributing electrical charge.

2. A composition according to claim 1 wherein at least one of R1, R2, R3, and R4 is independently any C1 to C6 alkyl group.

3. A composition according to claim 1, wherein at least one of R1, R2, R3, and R4 is independently selected from the group consisting of: hydrogen, methyl, and ethyl.

4. A composition according to claim 1 wherein X⁻ is selected from the group consisting of: chloride, bromide, iodide, nitrate, sulfate, hydrogen sulfate, phosphate, mono-hydrogen phosphate and poly-hydrogen phosphate, borate, silicate, bicarbonate, and alkyl carbonate ions.

5. A composition according to claim 1 wherein at least one of R1, R2, R3, and R4 present is sufficient for causing said material to have a visual appearance of any color selected from the group consisting of: red, yellow, blue, and combinations thereof, when said material is caused to be disposed on a substrate.

6. A combination useful for imparting coloration to a substrate, comprising:
a) at least one material according to claim 1;
b) a solvent; and
c) at least one ingredient selected from the group consisting of: dispersants, binders, biocides, humectants, mordants, chelating agents, viscosity modifiers, surfactants, fillers, defoamers, acrylic polymers, and non-acrylic polymers.

7. A combination according to claim 6, wherein said at least one material as defined in claim 1 is present in any amount in the range of between about 1% and 30% by weight based on the total weight of the combination.

8. A combination according to claim 6, wherein said combination is a solution and said solvent is present in any amount in the range of between about 50% to about 99.8% by weight based on the total weight of the combination.

9. A combination according to claim 6 wherein said solvent comprises water.

10. Method for imparting coloration to a substrate, comprising:
providing an aqueous solution comprising at least one material having the structure: 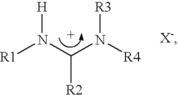
wherein at least one of R1, R2, R3, and R4 is a chromophore moiety, and non-chromophore moiety(ies) R1, R2, R3, and R4, when present, are each independently selected from the group consisting of: hydrogen and any C1-C24 hydrocarbyl group; and X⁻ is selected from the group consisting of: bicarbonate ion and any C1-C12 alkyl carbonate ion;
applying said aqueous solution to a substrate; and
drying the applied solution.

11. Method according to claim 10 wherein said aqueous solution is applied to a substrate by spraying.

12. Method according to claim 10 wherein said aqueous solution is applied to a substrate by an ink jet.

13. Method according to claim 10 wherein said substrate is selected from the group consisting of: plastics, paper, wood, fabrics, fibers and textiles.

14. A system suitable for dispensing a composition useful for imparting coloration to a substrate which comprises:
a) a pressurizable container capable of being at least partially-filled with a liquid solution, and having an outlet conduit;
b) an aqueous liquid solution comprising a material according to structure (II) of claim 1 wherein X⁻ is selected from the group consisting of: bicarbonate ion and any C1-C12 alkyl carbonate ion, disposed within said pressurizable container; and
c) a gas phase disposed within said container at a pressure greater than atmospheric pressure, said gas phase comprising carbon dioxide gas.

15. A system according to claim 14 wherein the gas phase is substantially-pure carbon dioxide gas.

16. A system according to claim 15 wherein the pressure of said substantially-pure carbon dioxide gas is any pressure in the range of between about 100 kiloPascals and about 42,000 kiloPascals, including all ranges therebetween.

17. A system according to claim 14 wherein the gas phase is a gas mixture comprising carbon dioxide and at least one other material which is a gas at standard temperature and pressure.

18. A system according to claim 17 wherein the partial pressure of carbon dioxide gas in said gas mixture is any partial pressure in the range of between about 10 kiloPascals and about 40,000 kiloPascals, including all ranges of pressures therebetween.

19. A system according to claim 17 wherein said gas mixture comprises nitrogen, at any partial pressure in the range of between about 10 kiloPascals and about 40,000 kiloPascals, including all ranges of pressures therebetween.

20. A method for dyeing an article comprising at least one material selected from the group consisting of: fabrics and fibers, comprising:
providing an aqueous solution comprising at least one material having the structure: 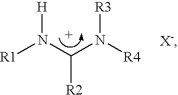
wherein at least one of R1, R2, R3, and R4 is a chromophore moiety, and non-chromophore moiety(ies) R1, R2, R3, and R4, when present, are each independently selected from the group consisting of: hydrogen and any C1-C24 hydrocarbyl group; and X⁻ is selected from the group consisting of: bicarbonate ion and any C1-C12 alkyl carbonate ion;
contacting said at least one material with said aqueous solution for a time sufficient to impart coloration to said at least one material;
optionally, rinsing said at least one material; and
drying said at least one material.
